(19) United States
(12) Patent Application Publication
(10) Pub. No.: US 2021/0300848 A1
(43) Pub. Date: Sep. 30, 2021

(54) CATALYSTS FOR PRODUCING ALCOHOLS AND ETHERS FROM SYNTHESIS GAS

(71) Applicant: SABIC Global Technologies B.V., Bergen op Zoom (NL)

(72) Inventors: Muhammad H. HAIDER, Riyadh (SA); Yasser T. AL-HARBI, Riyadh (SA); ahmed S. AL-ZENAIDI, Riyadh (SA)

(73) Assignee: SABIC Global Technologies B.V., Bergen op Zoom (NL)

(21) Appl. No.: 17/250,535

(22) PCT Filed: Sep. 16, 2019
(86) PCT No.: PCT/IB2019/057781

§ 371 Date: Feb. 1, 2021

Related U.S. Application Data
(60) Provisional application No. 62/732,227, filed on Sep. 17, 2018.

(51) Int. Cl. C07C 29/157; B01J 23/72; B01J 23/755; B01J 21/14; B01J 35/10; B01J 37/04; B01J 37/06; B01J 37/08; B01J 37/02; B01J 37/00; C07C 29/154; C07C 41/09

(52) U.S. Cl. CPC C07C 29/157; B01J 23/72; B01J 23/755; B01J 21/14; B01J 35/1019; C07C 41/09; B01J 37/06; B01J 37/082; B01J 37/0207; B01J 37/009; C07C 29/154; B01J 37/04

(57) ABSTRACT

Catalysts for the production of an alcohol and/or an ether from synthesis gas, methods of making the catalysts, and uses thereof are described. The catalyst can include catalytic Cu metal particles or oxides thereof and/or Ni metal particles or oxides thereof on an alkali metal or alkaline earth metal silicate support.

### CROSS REFERENCE TO RELATED APPLICATIONS

This application claims priority to and the benefit of U.S. Provisional Application No. 62/732,227, filed Sep. 17, 2018, the contents of which is incorporated into the present application by reference.

### BACKGROUND OF THE INVENTION

### A. Field of the Invention

The invention generally concerns catalysts for the production of alcohols and ethers from synthesis gas, methods of making the catalysts, uses thereof. The catalysts can include copper (Cu) particles, nickel (Ni) particles, or oxides thereof, or a combination thereof impregnated on an alkali metal or alkaline earth metal silicate support.

### B. Description of Related Art

The interest in converting synthesis gas (syngas) to alcohols is growing rapidly. Syngas is a mixture of carbon monoxide and hydrogen, with optional carbon dioxide can be obtained from various carbon-containing sources such as coal, natural gas, biomass, and as a by-product of various chemical production processes.

A variety of products, including paraffins, alcohols, olefins, and other chemicals can be obtained from the catalytic conversion of syngas. One significant syngas conversion route is via lower alcohol, i.e., C₃-C₄ alcohol or C₂-C₄ ether synthesis. Dimethyl ether (DME) is a special commodity chemical produced in two step process from syngas, which can then be used to produce C₂-C₄ olefins. Conventional production of DME includes a two-step process of producing methanol over a methanol synthesis catalyst and a second catalyst bed is used to dehydrate methanol to dimethyl ether as shown in reaction equations 1 and 2.

CO+2H₂↔CH₃OH ΔH⁰=−92.0 kJ mol⁻¹ (1)

2CH₃OH↔CH₃OCH₃+H₂O ΔH⁰=−23 kJ mol⁻¹ (2)

While various supported Cu and/or Ni catalysts are known, these catalyst suffer in high selectivity to carbon dioxide and/or methane and/or involve complicated methodology to prepare.

### SUMMARY OF THE INVENTION

A solution to at least some of the problems discussed above concerning production of ethers and alcohols from the catalytic conversion of syngas. The solution is premised on the production of dimethyl ether and/or methanol over single catalyst bed performing dual functions of producing and dehydrating methanol in single pass giving high selectivity towards desired products. The catalyst includes copper (Cu) particles and nickel (Ni) particles or oxides thereof, or mixtures thereof impregnated in an alkaline metal and/or alkaline earth metal silicate support. The produced ethers and alcohols (e.g., DME and methanol) can be directly converted to other products and/or sold. Other product include the production of olefins from dimethyl ether to olefins or methanol to olefins processes. Notably and as illustrated in a non-limiting manner in the Examples, the production of alcohol or ethers can be tuned based on the reaction temperatures. Thus, eliminating the need to use a two-step catalyst system for the production of ethers.

In one aspect of the current invention, catalysts capable of producing alcohols and ethers from synthesis gas are described. A catalyst can include Cu metal particles or oxides thereof, Ni metal particles or oxides thereof, or any combination thereof, impregnated in an alkali metal and/or alkaline earth metal silicate support. In a preferred embodiment, the support is an alkaline earth metal silicate. In one embodiment, the support is magnesia-silicate. The molar ratio of the alkaline earth metal to silicon oxide, preferably Mg:SiO₂, can be 10:90 to 40:60, preferably 25:75. The support can have a surface area from 100 to 300 m³/g. In some embodiments, the support does not include alumina. The catalyst does not require or include a phosphorous containing compound, a boron containing compound, a phosphorous and boron containing compound, a noble metal or compound thereof, zinc or a compound thereof or any combination thereof. The catalyst can include 0.01 wt. % to 5 wt. % Cu, more preferably 1.90 to 2 wt. % or about 1.95 wt. % Cu, and/or 0.01 wt. % to 15 wt. % Ni, preferably 3.9 to 4.0 wt. %, or about 3.95 wt. % Ni. In some aspects the catalyst includes Cu metal or oxides thereof and Ni metal or oxides thereof on a magnesia-silicate support. The catalyst does not include a NiCu alloy.

In another aspect of the invention, methods for preparing the catalyst of the present invention are described. A method can include the steps of: impregnating an alkali metal or alkaline earth metal silicate support with a Cu precursor material, a Ni precursor material or both under conditions sufficient to produce the catalyst of the present invention. The support can be obtained by contacting a solution that includes ammonia (e.g., 0.1 to 7 molar) and a alkali metal precursor material, a alkaline earth metal precursor material or both with SiO₂ under conditions sufficient to produce an alkali metal or alkaline earth metal silicate.

In yet another aspect of the present invention, processes to produce alcohols and/or ethers from synthesis gas are described. A process can include contacting a reactant feed that includes hydrogen (H₂) and carbon monoxide (CO) with the catalyst(s) of the present invention, under conditions sufficient to produce an alcohol (e.g., methanol, ethanol, propanol, or mixture thereof) and/or an ether (e.g., dimethyl ether). Conditions can include temperature (e.g., 230° C. to 310° C., preferably, 240° C. to 350° C.), weighted hourly space velocity (WHSV) (e.g., 1000 h⁻¹ to 3000 h⁻¹, preferably 1500 h⁻¹ to 2000 h⁻¹), pressure (e.g., 4.5 MPa to 5.5 MPa), or combinations thereof. At a temperature range of 230° C. to 280° C., alcohol production is preferred over ether production. At a temperature range of 285° C. to 310° C., ether production is preferred over alcohol production.

The following includes definitions of various terms and phrases used throughout this specification.

The terms “about” or “approximately” are defined as being close to as understood by one of ordinary skill in the art. In one non-limiting embodiment, the terms are defined to be within 10%, preferably within 5%, more preferably within 1%, and most preferably within 0.5%.

The terms “wt. %”, “vol. %”, or “mol. %” refers to a weight percentage of a component, a volume percentage of a component, or molar percentage of a component, respectively, based on the total weight, the total volume of material, or total moles, that includes the component. In a non-limiting example, 10 grams of component in 100 grams of the material is 10 wt. % of component.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the words “a” or “an” when used in conjunction with any of the terms “comprising,” “including,” “containing,” or “having” in the claims, or the specification, may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The catalysts of the present invention can “comprise,” “consist essentially of,” or “consist of” particular ingredients, components, compositions, etc. disclosed throughout the specification. With respect to the transitional phrase “consisting essentially of,” in one non-limiting aspect, a basic and novel characteristic of the catalysts of the present invention are their abilities to catalyze production of alcohols and ethers from synthesis gas in a one-step process.

In the context of the present invention, at least twenty embodiments are now described. Embodiment 1 is a catalyst for the conversion of synthesis gas to an alcohol and/or an ether. The catalyst includes copper (Cu) particles, nickel (Ni) particles, or oxides thereof, or a combination thereof, impregnated in an alkali metal and/or alkaline earth metal silicate support. Embodiment 2 is the catalyst of embodiment 1, wherein the support is the alkaline earth metal silicate. Embodiment 3 is the catalyst of any one of embodiments 1 to 2, wherein the support is a magnesia-silicate support. Embodiment 4 is the catalyst of any one of embodiments 1 to 3, wherein the catalyst does not include a phosphorous containing compound, a boron containing compound, a phosphorous and boron containing compound, a noble metal or compound thereof, zinc or a compound thereof, or a combination thereof. Embodiment 5 is the catalyst of any one of embodiments 1 to 4, comprising 0.01 wt. % to 5 wt. % Cu, preferably 1.90 to 2.0 wt. % Cu. Embodiment 6 is the catalyst of any one of embodiments 1 to 5, containing 0.01 wt. % to 15 wt. % Ni, preferably 3.9 to 4.0 wt. %. Embodiment 7 is the catalyst of any one of embodiments 1 to 6, wherein the catalyst includes Cu particles and Ni particles or oxides thereof. Embodiment 8 is the catalyst of any embodiment 7, wherein the catalyst does not include a NiCu alloy. Embodiment 9 is the catalyst of any one of embodiments 1 to 8, wherein a molar ratio of the alkaline earth metal to silicon oxide, preferably Mg:SiO₂, is 10:90 to 40:60, preferably 25:75. Embodiment 10 is the catalyst of any one of embodiments 1 to 9, wherein the support has a surface area from 100 to 300 m³/g. Embodiment 11 is the catalyst of any one of embodiments 1 to 10, wherein the support does not include alumina.

Embodiment 12 is a method of producing the catalyst of any one of embodiments 1 to 11 is the method includes the steps if impregnating an alkali metal or alkaline earth metal silicate support with a copper (Cu) precursor material, a nickel (Ni) precursor material or both under conditions sufficient to produce the catalyst. Embodiment 13 is the method of embodiment 12, wherein the support is obtained by contacting a solution comprising ammonia and an alkali metal precursor material, an alkaline earth metal precursor material, or both with SiO₂ under conditions sufficient to produce an alkali metal or alkaline earth metal silicate. Embodiment 14 is the method of any one of embodiments 12 to 13, wherein the ammonia concentration is 0.1 to 7 molar.

Embodiment 15 is a process to produce alcohols and/or ethers. The process includes the step of contacting a gaseous reactant stream comprising hydrogen H₂ and carbon monoxide (CO) with the catalyst of any one of embodiments 1-11 under reaction conditions suitable to produce an alcohol, an ether or both. Embodiment 16 is the process of embodiment 15, wherein the reaction conditions comprise a temperature of 230 to 280° C. and an alcohol is produced. Embodiment 17 is the process of embodiment 16, wherein the alcohol is methanol, ethanol, propanol or a mixture thereof. Embodiment 18 is the process of embodiment 15, wherein the reaction conditions comprise a temperature of 285° C. to 310° C. and an ether is produced. Embodiment 19 is the process of embodiment 18, wherein the ether is dimethyl ether. Embodiment 20 is the process of any one of embodiments 15 to 19, wherein the reaction conditions include a pressure of 4.5 to 5.5 MPa.

Other objects, features and advantages of the present invention will become apparent from the following detailed description, and examples. It should be understood, however, that the detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein. It is contemplated that any embodiment discussed herein can be implemented with respect to any method or composition of the invention, and vice versa. Furthermore, compositions of the invention can be used to achieve methods of the invention.

### DETAILED DESCRIPTION OF THE INVENTION

A discovery has been made that provides a solution to problems associated with catalysts used to produce alcohols and/or ethers from synthesis gas. The discovery is premised on using a catalyst that includes a Cu and/or Ni particles, or oxides thereof impregnated in an alkali metal or alkaline earth metal support. Such a catalyst allows for production of ethers in a one-step process, providing an economic advantage over conventional two-step ether production processes.

These and other non-limiting aspects of the present invention are discussed in further detail in the following sections.

### A. Catalysts of the Present Invention

The catalyst of the present invention can be a Cu and/or Ni metal or oxides thereof supported on an alkali metal or alkaline earth metal silicate support. The Cu or Ni supported catalyst can include at least, equal to or between any two of 0.01, 0.05, 0.1, 0.15, 0.5, 1.0. 1.5, 2.0, 2.5, 3.0, 3.5, 4.0, 4.5, and 5 wt. % of Cu, and/or at least, equal to or between any two of 0.01, 0.05, 0.1, 0.15, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10, 10.5, 11, 11.5, 12, 12.5, 13, 13.5, 14, 14.5, and 15 wt. % Ni. The catalyst of the present invention can include up to 20 wt. % of the total amount of total catalytic transition metal, from 0.001 wt. % to 20 wt. %, from 0.01 wt. % to 15 wt. %, or from 1 wt. % to 10 wt. % and all wt. % or at least, equal to, or between any two of 0.001 wt. %, 0.01 wt. %, 0.1 wt. %, 1 wt. %, 2 wt. %, 3 wt. %, 4 wt. %, 5 wt. %, 6 wt. %, 7 wt. %, 8 wt. %, 9 wt. %, 10 wt. %, 15 wt. %, and 20 wt. %, with the balance being support. For example, the catalyst can include 1.90 to 2.0 wt. % Cu, 3.9 to 4.0 wt. % Ni, and 94.0 to 94.1 wt. % alkaline earth metal silicate support (e.g., magnesia silicate support). In another example, the catalyst can include 0.1 to 5.0 wt. % Cu, 0.01 to 15 wt. % Ni, and 80.0 to 99.2 wt. % alkaline earth metal silicate support (e.g., magnesia silicate support). The support material can include alkali metal or alkaline earth metal silicates. Non-limiting examples of alkali metals (Column 1 of the Periodic Table) include lithium, sodium, potassium, rubidium, and cesium. Non-limiting examples of alkaline earth metals (Column 2 of the Periodic Table) include Mg, Ca, Sr, and Ba. In a preferred embodiment, the support material is magnesia silicate. The molar ratio of the alkali metal or alkaline earth metal to silicon oxide, can be at least, equal to, or between any two of 10:90, 15:85, 20:80, 25:75, 30:70, 35:65, and 40:60.

### B. Preparation of the Catalysts of the Present Invention

Methods for producing alcohols or dimethyl ether via a one-step process can include impregnating the alkali metal or alkaline earth metal silicate with a Cu or Ni precursor material.

### 1. Preparation of Support Material

The support material can be obtained by mixing silica with an aqueous solution of an alkali metal or alkaline earth metal precursor material (e.g., a magnesium salt) at 15 to 30° C. for 1 to 5 hours, or about 2 hours. Precursor materials can include chlorides, nitrates, sulfates or the like. In a preferred embodiments, MgCl₂ is used. In embodiments of the invention, the aqueous solution may have a metal (e.g., Mg⁺²) concentration in a range of 0.5 to 5 M and all ranges and values there between including ranges of 0.5 to 0.8 M, 0.8 to 1.1 M, 1.1 to 1.4 M, 1.4 to 1.7 M, 1.7 to 2.0 M, 2.0 to 2.3 M, 2.3 to 2.6 M, 2.6 to 2.9 M, 2.9 to 3.2 M, 3.2 to 3.5 M, 3.5 to 3.8 M, 3.8 to 4.1 M, 4.1 to 4.4 M, 4.4 to 4.7 M, and 4.7 to 5.0 M. In embodiments of the invention, the aqueous solution may have a silica concentration in a range of 40 to 90 wt. % and all ranges and values there between including ranges of 40 to 45 wt. %, 45 to 50 wt. %, 50 to 55 wt. %, 55 to 60 wt. %, 60 to 65 wt. %, 65 to 70 wt. %, 70 to 75 wt. %, 75 to 80 wt. %, 80 to 85 wt. %, and 85 to 90 wt. %. To this mixture, ammonia solution can be added and the solution agitated at 15 to 30° C. for 1 to 5 hours, or about 2 hours. The ammonia concentration can be at least, equal to, or between any two of 0.01, 0.1, 0.5, 1, 1.5, 2, 2.5, 3, 3.5, 4, 4.5, and 5 Molar. The resulting alkali metal or alkaline earth metal silicate can be separated using known separation techniques (e.g., filtration, centrifugation, etc.), optionally dried, and then calcined in the presence of an oxidizing source (e.g. air) at 300 to 600° C. and all ranges and values there between including ranges of 300 to 320° C., 320 to 340° C., 340 to 360° C., 360 to 380° C., 380 to 400° C., 400 to 420^{v}, 420 to 440° C., 440 to 460^{v}, 460 to 480° C., 480 to 500° C., 500 to 520° C., 520 to 540° C., 540 to 560° C., 560 to 580° C., and 580 to 600° C. A temperature ramp for the calcination may be in a range of 1 to 10 ° C./min⁻¹ and all ranges and values there between including 2° C./min⁻¹, 3° C./min⁻¹, 4° C./min⁻¹, 5° C./min⁻¹, 6° C./min⁻¹, 7° C./min⁻¹, 8° C./min⁻¹, and 9° C./min⁻¹.

### 2. Preparation of Catalyst of the Present Invention

The prepared support material can be impregnated at least one of a copper precursor salt and a nickel precursor salt to provide an impregnated catalytic precursor. Non-limiting examples of copper salts can include copper nitrate, copper sulfate, copper chloride, or combinations thereof. Non-limiting examples of nickel salts can include, nickel sulfate, nickel chloride, nickel nitrate, or combinations thereof. The copper and/or nickel concentration can be 0.01 to 1 M and all ranges and values there between including ranges of 0.01 to 0.05 M, 0.05 to 0.10 M, 0.10 to 0.15 M, 0.15 to 0.20 M, 0.20 to 0.25 M, 0.25 to 0.30 M, 0.30 to 0.35 M, 0.35 to 0.40 M, 0.40 to 0.45 M, 0.45 to 0.50 M, 0.50 to 0.55 M, 0.55 to 0.60 M, 0.60 to 0.65 M, 0.65 to 0.70 M, 0.70 to 0.75 M, 0.75 to 0.80 M, 0.80 to 0.85 M, 0.85 to 0.90 M, 0.90 to 0.95 M, and 0.95 to 1 M. The concentration can be sufficient to produce a catalyst having a total of 0.01 wt. % to 20 wt. % of catalytic metal. The resulting support material impregnated with catalytic material can be optionally dried for 1 to 24 hours (e.g., 1, 2, 3, 4, 5, 10, 12, 15, 20, 22, 24 hour or all values there between) at 100 to 125° C., or 110 to 120° C. or any range or value there between. The impregnated catalytic precursor can be calcined at a temperature of 300 to 600° C. and all ranges and values there between including ranges of 300 to 320° C., 320 to 340° C., 340 to 360° C., 360 to 380° C., 380 to 400° C., 400 to 420^{v}, 420 to 440° C., 440 to 460^{v}, 460 to 480° C., 480 to 500° C., 500 to 520° C., 520 to 540° C., 540 to 560° C., 560 to 580° C., and 580 to 600° C. A temperature ramp for the calcination may be in a range of 1 to 10° C./min⁻¹ and all ranges and values there between including 2° C./min⁻¹, 3° C./min⁻¹, 4° C./min⁻¹, 5° C./min⁻¹, 6° C./min⁻¹, 7° C./min⁻¹, 8° C./min⁻¹, and 9° C./min⁻¹. A time period for calcination can include 1 to 10 hours or 1, 2, 3, 4, 5, 6, 7, 8, 9, 10 hours.

The catalyst can be pelletized or shaped into a form suitable to be used in a catalytic bed. Binders and/or fillers can be used in the palletization process.

### C. Method of Producing an Alcohol or an Ether

The catalyst of the present invention can be used to produce an alcohol and/or ether from synthesis gas. The amount of alcohol and/or ether can be tuned based on the temperature of the reaction. The method can include obtaining a Cu and/or Ni metal supported catalyst of the present invention as described throughout the specification and examples. The catalyst of the present invention can be contacted with a reactant stream that includes H₂ and CO and optional CO₂ under reaction conditions sufficient to produce dimethyl ether and/or methanol. In a preferred embodiment the reactant stream is synthesis gas. Notably, the dimethyl ether is produced in a single-step process with a selectivity of. According to embodiments of the invention, the synthesis gas has a H₂ to CO volumetric ratio in a range of 1 to 3 and all ranges and values there between including ranges of 1 to 1.2, 1.2 to 1.4, 1.4 to 1.6, 1.6 to 1.8, 1.8 to 2.0, 2.0 to 2.2, 2.2 to 2.4, 2.4 to 2.6, 2.6 to 2.8, and 2.8 to 3.0.

Any type of reactor can be used. By way of example, a fixed bed reactor that includes the catalyst of the present invention in a fixed catalyst bed can be used. In another example, a fluidized bed reactor that includes catalyst of the present invention in a fluidized catalyst bed. In embodiments of the invention, the reaction conditions can include a reaction temperature in a range of 230 to 310° C. or at least, equal to, or between any two of 230° C., 240° C., 250° C., 260° C., 270° C., 280° C., 285° C., 290° C., 295° C., 300° C., 305° C., and 310° C. In embodiments, when more alcohol is desired, the temperature range can be 230° C. to 280° C., or any value or range there between. In embodiments that favor ether production, the temperature range can be 285 to 310° C.

In embodiments of the invention, the reaction conditions in block 202 can include a reaction pressure in a range of 4.5 to 7.0 MPa or at least, equal to, or between any two of 4.5, 5, 5.5, 6, 6.5, and 7 MPa. In a preferred instance, a pressure range of 4.5 to 5.5 MPa is used. In embodiments of the invention, the reaction conditions can also include a weight hourly space velocity in a range of 1500 to 2000 hr⁻¹ and all ranges and values there between including 1500 to 1525 hr⁻¹, 1525 to 1550 hr⁻¹, 1550 to 1575 hr⁻¹, 1575 to 1600 hr⁻¹, 1600 to 1625 hr⁻¹, 1625 to 1650 hr⁻¹, 1650 to 1675 hr⁻¹, 1675 to 1700 hr⁻¹, 1700 to 1725 hr⁻¹, 1725 to 1750 hr⁻¹, 1750 to 1775 hr⁻¹, 1775 to 1800 hr⁻¹, 1800 to 1825 hr⁻¹, 1825 to 1850 hr⁻¹, 1850 to 1875 hr⁻¹, 1875 to 1900 hr⁻¹, 1900 to 1925 hr⁻¹, 1925 to 1950 hr⁻¹, 1950 to 1975 hr⁻¹, 1975 to 2000 hr⁻¹.

The products produced can include an alcohol and/or an ether or mixtures thereof. Non-limiting examples of alcohols include methanol, propanol, iso-propanol, ethanol, butanol or mixtures thereof. In a preferred embodiment, methanol is produced. Non-limiting examples of ether compounds include dimethyl ether, diethyl ether, dipropyl ether or mixtures thereof. Mixed ethers can also be produced. In a preferred embodiment dimethyl ether is produced.

### EXAMPLES

The present invention will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results.

### Materials Used

### Example 1

### Preparation of Catalyst A-K

Catalyst A. A magnesium chloride (MgCl₂) solution (4.18 g in 100 ml distilled water) was mixed with silica (3 g in 50 ml distilled water). After mixing for 2 h, 5 M ammonia solution (200 ml) was added and the mixture was stirred for 2 h. The mixture was then filtered to collect the solid. The collected solid was then washed with hot water, and dried overnight. The dried solid was then calcined at 400° C. in air at the ramp rate of 5° C.min−1 for 4 hr. Material obtained was impregnated with copper (0.31 g from nitrate in 50 ml distilled water) for 4 hr followed by drying overnight at 120° C. to obtain a catalyst precursor. The catalyst precursor was subsequently calcined in static air in a furnace at 500° C. with a temperature ramp of 5° C. min−1 for 5 hr to produce catalyst A.

Catalyst B. A MgCl₂ solution (4.18 g in 100 ml distilled water) was mixed with silica (3 g in 50 ml distilled water). After mixing for 2 hr, 5 M ammonia solution (200 ml) was added and the mixture was stirred for 2 hr. The mixture was then filtered, washed with hot water, and dried overnight to obtain a solid. The solid was then calcined at 400° C. in air at the ramp rate of 5° C. min−1 for 4 hr. Material obtained was impregnated with nickel (0.84 g nickel nitrate in 50 ml distilled water) for 4 hr followed by drying overnight at 120° C. to obtain a catalyst precursor. The catalyst precursor was subsequently calcined in static air in the furnace (500° C./5° C. min−1, 5 hr) to obtain Catalyst B.

Catalyst C. A MgCl₂ solution (4.18 g in 100 ml distilled water) was mixed with silica (3 g in 50 ml distilled water). After mixing for 2 hr, 5 M ammonia solution (200 ml) was added and the mixture was stirred for 2 hr. The mixture was then filtered, washed with hot water, and dried overnight to obtain a solid. The solid was then calcined at 400° C. in air at the ramp rate of 5° C. min−1 for 4 hr. Material obtained was impregnated with nickel (0.84 g nickel nitrate in 50 ml distilled water) and copper (0.31 g copper nitrate in 50 ml distilled water) for 4 hr followed by drying overnight at 120° C. to obtain a catalyst precursor. The catalyst precursor was subsequently calcined in static air in the furnace (500° C./5° C. min−1, 5 hr) to obtain Catalyst C.

Catalyst D. A MgCl₂ solution (4.18 g in 100 ml distilled water) was mixed with silica (3 g in 50 ml distilled water). After mixing for 2 hr, 7 M ammonia solution. (200 ml) was added and the mixture was stirred for 2 hr. The mixture was then filtered, washed with hot water, and dried overnight to obtain a solid. The solid was then calcined at 400° C. in air at the ramp rate of 5° C. min−1 for 4 hr. Material obtained was impregnated with copper (0.31 g copper nitrate in 50 ml distilled water) for 4 hr followed by drying overnight at 120° C. to obtain a catalyst precursor. The catalyst precursor was subsequently calcined in static air in the furnace (500° C./5° C. min−1, 5 hr) to obtain Catalyst D.

Catalyst E. A MgCl₂ solution (4.18 g in 100 ml distilled water) was mixed with silica (3 g in 50 ml distilled water). After mixing for 2 hr, 7 M ammonia solution (200 ml) was added and the mixture was stirred for 2 hr. The mixture was filtered, washed with hot water, and dried overnight before calcination at 400° C. in air at the ramp rate of 5° C. min−1 for 4 h. Material obtained was impregnated with nickel (0.84 g nickel nitrate in 50 ml distilled water) for 4 hr followed by drying overnight at 120° C. to obtain a catalyst precursor. The catalyst precursor was subsequently calcined in static air in the furnace (500° C./5° C. min−1, 5 h) to obtain Catalyst E.

Catalyst F. A MgCl₂ solution (4.18 g in 100 ml distilled water) was mixed with silica (3 g in 50 ml distilled water). After mixing for 2 hr, 7 M ammonia solution (200 ml) was added and the mixture was stirred for 2 hr. After stirring, the mixture was filtered and washed with hot water, and dried overnight to obtain a solid. The solid was then calcined at 400° C. in air at the ramp rate of 5° C. min−1 for 4 hr. Material obtained was impregnated with nickel (0.84 g nickel nitrate in 50 ml distilled water) and copper (0.31 g copper nitrate in 50 ml distilled water) for 4 hr followed by drying overnight at 120° C. to obtain a catalyst precursor. The catalyst precursor was subsequently calcined in static air in the furnace (500° C./5° C. min−1, 5 h) to obtain Catalyst F.

Catalyst G. A MgCl₂ solution (4.18 g in 100 ml distilled water) was mixed with silica (3 g in 50 ml distilled water). After mixing for 2 hr, 1 M ammonia solution (200 ml) was added and the mixture was stirred for 2 hr. After stirring, the mixture was filtered and washed with hot water, and dried overnight to obtain a solid. The solid was then calcined at 400° C. in air at the ramp rate of 5° C. min−1 for 4 hr. Material obtained was impregnated with copper (0.31 g copper nitrate in 50 ml distilled water) for 4 hr followed by drying overnight at 120° C. to obtain a catalyst precursor. The catalyst precursor was subsequently calcined in static air in the furnace (500° C./5° C. min−1, 5 hr) to obtain Catalyst G.

Catalyst H. A MgCl₂ solution (4.18 g in 100 ml distilled water) was mixed with silica (3 g in 50 ml distilled water). After mixing for 2 hr, 1 M ammonia solution (200 ml) was added and the mixture was stirred for 2 hr. After stirring, the mixture was filtered, washed with hot water, and dried overnight to obtain a solid. The solid was calcined at 400° C. in air at the ramp rate of 5° C. min−1 for 4 hr. Material obtained was impregnated with nickel (0.84 g nickel nitrate in 50 ml distilled water) for 4 hr followed by drying overnight at 120° C. to obtain a catalyst precursor. The catalyst precursor was subsequently calcined in static air in the furnace (500° C./5° C. min−1, 5 hr) to obtain Catalyst H.

Catalyst I. A MgCl₂ solution (4.18 g in 100 ml distilled water) was mixed with silica (3 g in 50 ml distilled water). After mixing for 2 hr, 1 M ammonia solution (200 ml) was added and the mixture was stirred for 2 hr. After stirring, the mixture was filtered, washed with hot water, and dried overnight to obtain a solid. The solid was then calcined at 400° C. in air at the ramp rate of 5° C. min−1 for 4 hr. Material obtained was impregnated with nickel (0.84 g nickel nitrate in 50 ml distilled water) and copper (0.31 g copper nitrate in 50 ml distilled water) for 4 hr followed by drying overnight at 120° C. to obtain a catalyst precursor. The catalyst precursor was subsequently calcined in static air in the furnace (500° C./5° C. min−1, 5 hr) to obtain Catalyst I.

Catalyst J. A MgCl₂ solution (4.18 g in 100 ml distilled water) was mixed with silica (3 g in 50 ml distilled water). After mixing for 2 hr, 5 M ammonia solution (200 ml) was added and the mixture was stirred for 6 hr. After stirring, the mixture was filtered and washed with hot water, and dried overnight to obtain a solid. The solid was then calcined at 400° C. in air at the ramp rate of 5° C. min−1 for 4 hr. Material obtained was impregnated with copper (0.31 g copper nitrate in 50 ml distilled water) for 4 hr followed by drying overnight at 120° C. to obtain a catalyst precursor. The catalyst precursor was subsequently calcined in static air in the furnace (500° C./5° C. min−1, 5 hr) to obtain Catalyst J.

Catalyst K. A MgCl₂ solution (4.18 g in 100 ml distilled water) was mixed with silica (3 g in 50 ml distilled water). After mixing for 2 hr, 5 M ammonia solution. (200 ml) was added and the mixture was stirred for 6 hr. After stirring, the mixture was filtered, washed with hot water, and dried overnight to obtain a solid. The solid was then calcined at 400° C. in air at the ramp rate of 5° C. min−1 for 4 hr. Material obtained was impregnated with nickel (0.84 g nickel nitrate in 50 ml distilled water) for 4 hr, followed by drying overnight at 120° C. to obtain a catalyst precursor. The catalyst precursor was subsequently calcined in static air in the furnace (500° C./5° C. min−1, 5 hr) to obtain Catalyst K.

### Example 2

### Catalyst Evaluation

Each of Catalysts A-K was evaluated for the activity, selectivity, and short term and long term stability. Prior to evaluation, all of the catalysts were subjected to activation procedure under the activation conditions including a temperature of 350° C. with a ramp rate of 3° C. min⁻¹ for 16 hr by 50:50 H₂/N₂ flow. The weight hourly space velocity during the activation was 3600 h⁻¹. Catalytic evaluation was carried out in a high throughput fixed bed flow reactor setup, which was housed in a temperature controlled system fitted with regulators to maintain target pressure during the reaction. The products of the reactions were analyzed through online gas chromatography (GC) analysis.

After activation, each of Catalysts A-K was used for production of dimethyl ether and methanol from synthesis gas (syngas) in the single-step production process, according to embodiments of the invention. Catalyst J includes only Cu and catalyst K included only Ni. The target products including methanol and dimethyl ether and side products including methane and carbon dioxide were analyzed. Both of the products are produced in good selectivities over catalysts A-I, along with side products of carbon dioxide and methane. At pressures less than 7 MPa, the side product formation was reduced. In contrast catalyst J and K had low to zero DME selectivity and produced mostly methane and/or paraffins. Thus, the single catalyst performed both methanol formation and methanol dehydration functions as seen in product distributions where methanol and dimethyl ether are produced side by side. The results are tabulated in Table 1.

Although embodiments of the present application and their advantages have been described in detail, it should be understood that various changes, substitutions and alterations can be made herein without departing from the spirit and scope of the embodiments as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the above disclosure, processes, machines, manufacture, compositions of matter, means, methods, or steps, presently existing or later to be developed that perform substantially the same function or achieve substantially the same result as the corresponding embodiments described herein can be utilized. Accordingly, the appended claims are intended to include within their scope such processes, machines, manufacture, compositions of matter, means, methods, or steps.

TABLE 1

| | Temperature (° C.) | | | | | | | | | | |
|---|---|---|---|---|---|---|---|---|---|---|---|
| | 250/300° C. at 5.0 MPa | | | | | | | | | | |
| Catalysts | A | B | C | D | E | F | G | H | I | J | K |
| Conversion | 0.2/2 | 0.2/0.7 | 0/3 | 0.2/2.4 | 0.5/11 | 0.4/23 | 0/4 | 0.3/46 | 0.1/4.5 | 0/3 | 5/57 |
| (mol. %) | | | | | | | | | | | |
| Selectivities | | | | | | | | | | | |
| (mol. %) | | | | | | | | | | | |
| Alcohols (total) | 55/27 | 0/8 | 0/32 | 32/21 | 0/4 | 40/19 | 0/26 | 0/2 | 50/50 | 0/33 | 0/2 |
| a) MeOH | 55/27 | 0/8 | 0/32 | 32/21 | 0/3 | 40/18 | 0/26 | 0/1 | 50/50 | 0/29 | 0/0.5 |
| b) EtOH | 0/0 | 0/0 | 0/0 | 0/0 | 0/1 | 0/1 | 0/0 | 0/0.5 | 0/0 | 0/4 | 0/1 |
| c) PrOH | 0/0 | 0/0 | 0/0 | 0/0 | 0/0 | 0/0 | 0/0 | 0/0.5 | 0/0 | 0/0 | 0/0.5 |
| DME | 25/42 | 48/0 | 0/20 | 0/40 | 0/0 | 17/5 | 0/49 | 0/0 | 20/31 | 0/11 | 0/0 |
| Olefins | 0/0 | 0/0 | 0/0 | 0/0 | 0/0 | 0/0.2 | 0/0 | 0/0.1 | 0/0 | 0/0 | 0/0.1 |
| Paraffins (C₂₊) | 6/10 | 0/0 | 0/2 | 19/12 | 0/7 | 0/4 | 0/1 | 0/10 | 0/3 | 0/31 | 0/10 |
| Methane | 14/8 | 52/78 | 0/40 | 18/13 | 100/73 | 41/55 | 0/3 | 100/70 | 33/10 | 0/11 | 100/78 |
| CO₂ | 0/10 | 0/14 | 0/6 | 0/15 | 0/13 | 0/15 | 0/20 | 0/20 | 0/10 | 0/14 | 0/10 |

| | Temperature (° C.) | | | | | | | | | | |
|---|---|---|---|---|---|---|---|---|---|---|---|
| | 250/300° C. at 7.0 MPa | | | | | | | | | | |
| Catalysts | A | B | C | D | E | F | G | H | I | J | K |
| Conversion | 1/3 | 0.5/1.3 | 1/4 | 1/4 | 1/12 | 0.4/26 | 1/5 | 1/52 | 1/6 | 1/4 | 5/65 |
| (mol. %) | | | | | | | | | | | |
| Selectivities | | | | | | | | | | | |
| (mol. %) | | | | | | | | | | | |
| Alcohols (total) | 41/21 | 0/5 | 12/30 | 15/18 | 0/3 | 50/21 | 15/24 | 7/2 | 10/47 | 15/33 | 2/1 |
| a) MeOH | 41/21 | 0/5 | 12/30 | 15/18 | 0/3 | 50/20 | 15/24 | 7/1 | 10/46 | 15/28 | 2/0.8 |
| b) EtOH | 0/0 | 0/0 | 0/0 | 0/0 | 0/1 | 0/1 | 0/0 | 0/0.8 | 0/1 | 0/5 | 0/0.2 |
| c) PrOH | 0/0 | 0/0 | 0/0 | 0/0 | 0/0 | 0/0 | 0/0 | 0/0.1 | 0/0 | 0/0 | 0/0.5 |
| DME | 18/37 | 0/0 | 8/21 | 0/38 | 0/0 | 25/6 | 19/50 | 0/0 | 9/32 | 9/9 | 0/0 |
| Olefins | 0/0 | 0/0 | 0/0 | 0/0 | 0/0 | 0/0.3 | 0/0 | 0/0.1 | 0/0 | 0/0 | 0/0.1 |
| Paraffins (C₂₊) | 10/10 | 0/0 | 0/2 | 10/12 | 0/7 | 0/4 | 0/1 | 0/10 | 0/3 | 0/20 | 2/10 |
| Methane | 20/6 | 95/75 | 75/30 | 75/7 | 95/83 | 25/50 | 60/3 | 85/77 | 75/10 | 70/9 | 95/75 |
| CO₂ | 0/24 | 0/15 | 0/20 | 0/58 | 0/7 | 0/22 | 0/20 | 0/9 | 0/8 | 0/27 | 0/11 |

1. A catalyst for the conversion of synthesis gas to an alcohol and/or an ether, the catalyst comprising copper (Cu) particles, nickel (Ni) particles, or oxides thereof, or a combination thereof, impregnated in an alkali metal and/or alkaline earth metal silicate support.

2. The catalyst of claim 1, wherein the support is the alkaline earth metal silicate.

3. The catalyst of claim 1, wherein the support is a magnesia-silicate support.

4. The catalyst of claim 1, wherein the catalyst does not include a phosphorous containing compound, a boron containing compound, a phosphorous and boron containing compound, a noble metal or compound thereof, zinc or a compound thereof, or a combination thereof.

5. The catalyst of claim 1, comprising 0.01 wt. % to 5 wt. % Cu.

6. The catalyst of claim 1, comprising 0.01 wt. % to 15 wt. % Ni, preferably 3.9 to 4.0 wt. %

7. The catalyst of claim 1, wherein the catalyst comprises Cu particles and Ni particles or oxides thereof.

8. The catalyst of any claim 7, wherein the catalyst does not include a NiCu alloy,

9. The catalyst of claim 1, wherein a molar ratio of the alkaline earth metal to silicon oxide, preferably Mg:SiO₂, is 10:90 to 40:60.

10. The catalyst of claim 1, wherein the support has a surface area from 100 to 300 m³/g.

11. The catalyst of claim 1, wherein the support does not include alumina.

12. The catalyst of claim 1, the method comprising impregnating an alkali metal or alkaline earth metal silicate support with a copper (Cu) precursor material, a nickel (Ni) precursor material or both under conditions sufficient to produce the catalyst.

13. The method of claim 12, wherein the support is obtained by contacting a solution comprising ammonia and an alkali metal precursor material, an alkaline earth metal precursor material, or both with SiO₂ under conditions sufficient to produce an alkali metal or alkaline earth metal silicate.

14. The catalyst of claim 12, wherein the ammonia concentration is 0.1 to 7 molar.

15. A process to produce alcohols and/or ethers, the process comprising contacting a gaseous reactant stream comprising hydrogen H₂ and carbon monoxide (CO) with the catalyst of claim 1 under reaction conditions suitable to produce an alcohol, an ether or both.

16. The process of claim 15, wherein the reaction conditions comprise a temperature of 230 to 280° C. and an alcohol is produced.

17. The process of claim 16, wherein the alcohol is methanol, ethanol, propanol or a mixture thereof.

18. The process of claim 15, wherein the reaction conditions comprise a temperature of 285° C. to 310° C. and an ether is produced.

19. The process of claim 18, wherein the ether is dimethyl ether.

20. The catalyst of claim 15, wherein the reaction conditions comprise a pressure of 4.5 to 5.5 MPa.
